(19) United States
(12) Patent Application Publication
(10) Pub. No.: US 2016/0199873 A1
(43) Pub. Date: Jul. 14, 2016

(54) PARTICLE SUPPLYING APPARATUS

(71) Applicant: LIVEDO CORPORATION, Ehime (JP)

(72) Inventor: Kazuya MARUHATA, Tokushima (JP)

(21) Appl. No.: 14/914,116

(22) PCT Filed: Aug. 7, 2014
(86) PCT No.: PCT/JP2014/070835

§ 371 Date: Feb. 24, 2016

(30) Foreign Application Priority Data
Aug. 30, 2013 (JP) 2013-179586

(51) Int. Cl. B05C 19/04; B05C 19/06; A61F 13/15

(52) U.S. Cl. CPC B05C 19/04; A61F 13/15642; B05C 19/06; A61F 2013/15821

(57) ABSTRACT

A particle supplying apparatus includes a cylinder part (21) having a plurality of recessed portion rows (213) arranged along a cylinder rotation axis (R1), each of the recessed portion rows being recessed portions (212) arrayed at a fixed pitch in a circumferential direction in a cylinder outer surface (211). Above the cylinder part is a particle filling opening through which particles of an absorbent material is successively applied to the plurality of recessed portions. Part of the cylinder outer surface is covered by a cover part that extends from the particle filling opening to a lower portion of the cylinder part in a rotation direction, and particles are successively emitted from the plurality of recessed portions in the vicinity of the front edge of the cover part in the rotation direction.

### TECHNICAL FIELD

The present invention relates to a particle supplying apparatus for supplying particles of an absorbent material or a deodorant material onto a sheet member.

### BACKGROUND ART

Absorbent articles such as absorbent pads for light incontinence, which are used by being attached to the inner sides of disposable diapers, conventionally use absorbent sheets produced by sandwiching and fixing particles of a highly absorbent resin or other materials between two sheet members of a nonwoven fabric, for example. When manufacturing such absorbent sheets, particles of a highly absorbent resin are supplied onto one sheet member that is being conveyed at a constant speed, and then the other sheet member is layered on and bonded to the one sheet member.

Japanese Patent Application Laid-Open No. 2013-17563 (Document 1) discloses a technique for ejecting particles of a highly absorbent resin onto a sheet member by rotating a cylinder part that has a plurality of recessed portions in the outer surface. More specifically, a particle filling part for successively filling a plurality of circumferentially arrayed recessed portions with particles is provided above the cylinder part. A first cover part that extends from the particle filling part forward in the rotation direction of the cylinder part is further provided, and each recessed portion that has passed the particle filling part is covered with the first cover part until the recessed portion reaches in the vicinity of the lowermost portion of the cylinder part. When each recessed portion is passing over the front edge of the first cover part, particles in the recessed portion is ejected onto the sheet member. The cylinder part of Document 1 has a plurality of recessed portion rows arranged in the axial direction, each of the recessed portion rows being a plurality of recessed portions arrayed circumferentially at the same axial position. This configuration enables manufacture of absorbent sheets with strip-like particle existence regions.

The above-described apparatus including the cylinder part, the particle filling part, and the cover part allows a certain amount of particles to be retained in the recessed portions by leveling off the particles applied by the particle filling part to the recessed portions along the rear edge of the cover part in the rotation direction. However, if particles are caught between the rear edge and dividers that each divide two circumferentially adjacent recessed portions, the particles are crushed and an impact is given to the cylinder part and the cover part. If particles are caught simultaneously in several recessed portion rows, a strong impact will be given to the entire apparatus.

### SUMMARY OF INVENTION

The present invention is intended for a particle supplying apparatus for supplying particles of an absorbent material or a deodorant material onto a sheet member, and it is an object of the present invention to reduce an impact that may be caused by particles getting caught between dividers in a plurality of recessed portion rows and an edge of a cover part.

One preferable particle supplying apparatus according to the present invention includes a cylinder part of a generally cylindrical shape that rotates about a rotation axis pointing in a horizontal direction and has a plurality of recessed portion rows arranged along the rotation axis, each of the plurality of recessed portion rows being a plurality of recessed portions arrayed at a fixed pitch in a circumferential direction in an outer surface of the cylinder part, a particle filling part for housing particles of an absorbent material or a deodorant material above the cylinder part and successively filling the plurality of recessed portions in each of the plurality of recessed portion rows with particles at a particle filling opening that opposes the outer surface of the cylinder part, and a half cover part that extends in a rotation direction from the particle filling opening to a lower portion of the cylinder part and covers part of the outer surface of the cylinder part to allow particles to be successively emitted in the vicinity of a front edge of the half cover part in the rotation direction from the plurality of recessed portions toward a sheet member that is being conveyed under the cylinder part. The plurality of recessed portion rows are three or more recessed portion rows, and when a portion of the cylinder part that divides two recessed portions adjacent to each other in the circumferential direction is referred to as a divider, the plurality of recessed portion rows include at least three recessed portion rows among which positions of dividers in the circumferential direction differ.

According to the present invention, it is possible to reduce an impact that may be caused by particles getting caught between the dividers in the plurality of recessed portion rows and the rear edge of the half cover part in the rotation direction.

More preferably, the positions of dividers in the circumferential direction differ among all of the plurality of recessed portion rows. With this configuration, it is possible to further reduce the impact.

Another preferable particle supplying apparatus according to the present invention includes a cylinder part of a generally cylindrical shape that rotates about a rotation axis pointing in a horizontal direction and has a plurality of recessed portion rows arranged along the rotation axis, each of the plurality of recessed portion rows being a plurality of recessed portions arrayed at a fixed pitch in a circumferential direction in an outer surface of the cylinder part, a particle filling part for housing particles of an absorbent material or a deodorant material above the cylinder part and successively filling the plurality of recessed portions in each of the plurality of recessed portion rows with particles at a particle filing opening that opposes the outer surface of the cylinder part, and a half cover part that extends in a rotation direction from the particle filling opening to a lower portion of the cylinder part and covers part of the outer surface of the cylinder part to allow particles to be successively emitted in the vicinity of a front edge of the half cover part in the rotation direction from the plurality of recessed portions toward a sheet member that is being conveyed under the cylinder part. When a portion of the cylinder part that divides two recessed portions adjacent to each other in the circumferential direction is referred to as a divider, each divider extends in a direction inclined with respect to the rotation axis on the outer surface of the cylinder part. With this configuration, it is possible to reduce an impact that may be caused by particles getting caught between the dividers in the plurality of recessed portion rows and the rear edge of the half cover part in the rotation direction.

Yet another preferable particle supplying apparatus according to the present invention includes a cylinder part of a generally cylindrical shape that rotates about a rotation axis pointing in a horizontal direction and has a plurality of recessed portion rows arranged along the rotation axis, each of the plurality of recessed portion rows being a plurality of recessed portions arrayed at a fixed pitch in a circumferential direction in an outer surface of the cylinder part, a particle filling part for housing particles of an absorbent material or a deodorant material above the cylinder part and successively filling the plurality of recessed portions in each of the plurality of recessed portion rows with particles at a particle filling opening that opposes the outer surface of the cylinder part, and a half cover part that extends in a rotation direction from the particle filling opening to a lower portion of the cylinder part and covers part of the outer surface of the cylinder part to allow particles to be successively emitted in the vicinity of a front edge of the half cover part in the rotation direction from the plurality of recessed portions toward a sheet member that is being conveyed under the cylinder part. When a portion of the cylinder part that divides two recessed portions adjacent to each other in the circumferential direction is referred to as a divider, each divider has a surface that is located closer to the rotation axis than a region between the plurality of recessed portion rows is on the outer surface of the cylinder part. With this configuration, it is possible to reduce an impact that may be caused by particles getting caught between the dividers in the plurality of recessed portion rows and the rear edge of the half cover part in the rotation direction.

These and other objects, features, aspects and advantages of the present invention will become more apparent from the following detailed description of the present invention when taken in conjunction with the accompanying drawings.

### BRIEF DESCRIPTION OF DRAWINGS

FIG. 1 illustrates a configuration of an absorbent sheet manufacturing apparatus according to a first embodiment;

FIG. 2 is a cross-sectional view of the configuration in the vicinity of a cylinder part;

FIG. 3 illustrates a cylinder outer surface of the cylinder part;

FIG. 4 is a bottom view of a particle supplying apparatus;

FIG. 5 illustrates a cylinder part according to a comparative example;

FIG. 6 illustrates another example of the cylinder part;

FIG. 7 illustrates a cylinder part according to a second embodiment;

FIG. 8 illustrates another example of the cylinder part;

FIG. 9 illustrates a particle supplying apparatus according to a third embodiment;

FIG. 10 illustrates another example of the particle supplying apparatus;

FIG. 11 illustrates another example of the particle supplying apparatus;

FIG. 12 illustrates another example of the cylinder part; and

FIG. 13 illustrates another example of the cylinder part.

### DESCRIPTION OF EMBODIMENTS

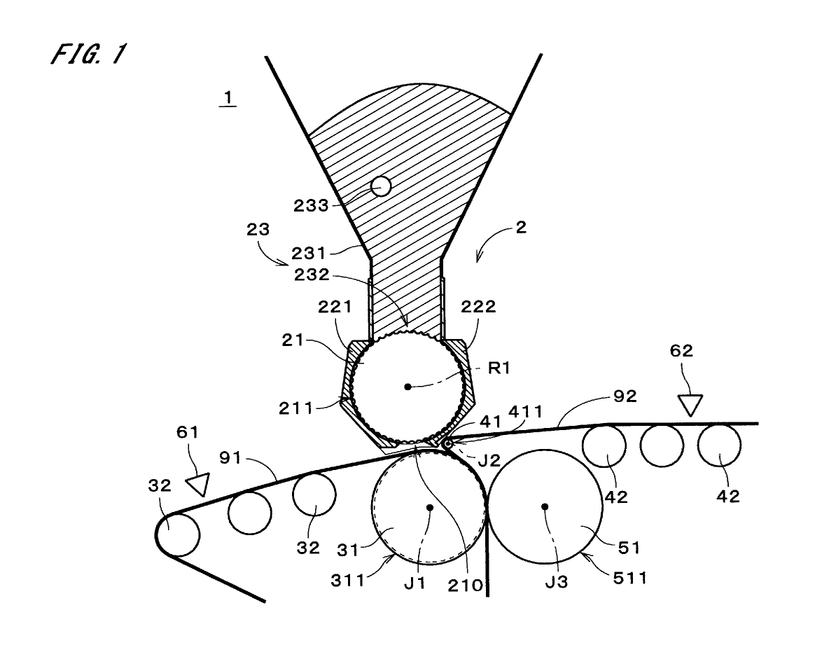

FIG. 1 illustrates a configuration of an absorbent sheet manufacturing apparatus 1 according to a first embodiment of the present invention. The absorbent sheet manufacturing apparatus 1 is one sheet member manufacturing apparatus for absorbent articles and manufactures absorbent sheets by putting particles of an absorbent material between sheet members of, for example, a nonwoven fabric. The absorbent sheets are absorbent-article sheet members usable in absorbent articles such as disposable diapers and absorbent pads for light incontinence.

Examples of particles of the absorbent material in the absorbent sheet manufacturing apparatus 1 include particles of crosslinked materials of partially neutralized polyacrylic acids, hydrolysates of starch-acrylic acid graft polymers, saponified materials of vinyl acetate-acrylic ester copolymers, hydrolysates of acrylonitrile copolymers or acrylamide copolymers or crosslinked materials of these copolymers, crosslinked materials of cationic monomers, and crosslinked materials of polyamino acids.

The absorbent sheet manufacturing apparatus 1 includes a cylinder part 21 that is a generally cylindrical member centered on a rotation axis R1 (hereinafter, referred to as a “cylinder rotation axis R1”) pointing in a horizontal direction, a first sheet conveying roller 31 of a generally columnar shape centered on a first central axis J1 pointing in a direction (hereinafter, referred to as an “axial direction”) parallel to the cylinder rotation axis R1, a second sheet conveying roller 41 of a generally columnar shape centered on a second central axis J2 pointing in the axial direction, and a bonding roller 51 of a generally columnar shape centered on a third central axis J3 pointing in the axial direction. For ease of understanding of the drawing, cross-sections of the cylinder part 21 and each roller are not diagonally hatched in FIG. 1 (the same applies to the other drawings).

The cylinder part 21, the first sheet conveying roller 31, and the bonding roller 51 have approximately the same diameter. The diameter of the second sheet conveying roller 41 is smaller than the diameters of the cylinder part 21, the first sheet conveying roller 31, and the bonding roller 51. The cylinder part 21 has a cylinder outer surface 211 that is a generally cylindrical surface centered on the cylinder rotation axis R1. The first sheet conveying roller 31 has a first roller outer surface 311 that is a generally cylindrical surface centered on the first central axis J1. The second sheet conveying roller 41 has a second roller outer surface 411 that is a generally cylindrical surface centered on the second central axis J2. The bonding roller 51 has a bonding roller outer surface 511 that is a generally cylindrical surface centered on the third central axis J3.

The first sheet conveying roller 31 is located below the cylinder part 21 in such a way that the lowermost part of the cylinder part 21 and the uppermost part of the first sheet conveying roller 31 are in close proximity to each other. The first central axis J1 of the first sheet conveying roller 31 is located to the right of the cylinder rotation axis R1 of the cylinder part 21 in FIG. 1. The second sheet conveying roller 41 is located in close proximity to the cylinder part 21 and the first sheet conveying roller 31 to the right of the lowermost part of the cylinder part 21 and the uppermost part of the first sheet conveying roller 31 in FIG. 1. More specifically, the second central axis J2 and the entire second sheet conveying roller 41 are located between the cylinder outer surface 211 and the first roller outer surface 311 that face each other in the up-down direction, to the right of the lowermost part of the cylinder part 21 and the uppermost part of the first sheet conveying roller 31 in FIG. 1. The bonding roller 51 is located adjacent to the right of the first sheet conveying roller 31 in FIG. 1.

The absorbent sheet manufacturing apparatus 1 further includes a plurality of auxiliary rollers 32 and 42, each having a generally columnar shape centered on a central axis pointing in the axial direction, and first and second application parts 61 and 62 for applying an adhesive (in the present embodiment, a hot-melt adhesive). The plurality of auxiliary rollers 32 and the first application part 61 are located to the left of the cylinder part 21 and the first sheet conveying roller 31 in FIG. 1. The plurality of auxiliary rollers 42 and the second application part 62 are located to the right of the bonding roller 51 in FIG. 1.

The cylinder part 21 rotates counterclockwise in FIG. 1, which is a predetermined rotation direction, about the cylinder rotation axis R1. The cylinder part 21 supplies particles of an absorbent material (e.g., particles of a highly absorbent resin such as a super absorbent polymer (SAP); hereinafter, simply referred to as “particles”) onto a first sheet member 91 in the vicinity of the lowermost part of the cylinder part 21, the first sheet member being a continuous sheet made of, for example, a nonwoven fabric. The first sheet conveying roller 31 rotates in the opposite direction to the rotation direction of the cylinder part 21 (i.e., clockwise in FIG. 1) about the first central axis J1 to convey the first sheet member 91 along the first roller outer surface 311 to the vicinity of the lowermost portion of the cylinder part 21.

The second sheet conveying roller 41 rotates in the same direction as the rotation direction of the cylinder part 21 (i.e., counterclockwise in FIG. 1) about the second central axis J2 in synchronization with the rotation of the first sheet conveying roller 31. The second sheet conveying roller 41 conveys a second sheet member 92, which is a continuous sheet made of, for example, a nonwoven fabric, along the second roller outer surface 411 to the vicinity of the lowermost portion of the cylinder part 21. As described above, the second sheet conveying roller 41 is located to the right of the lowermost portion of the cylinder part 21 in FIG. 1, i.e., forward of the lowermost portion of the cylinder part 21 in the rotation direction of the cylinder part 21. The second sheet conveying roller 41 is also located forward of the uppermost portion of the first sheet conveying roller 31 in the rotation direction of the first sheet conveying roller 31. The second sheet member 92 that is conveyed to the vicinity of the lowermost portion of the cylinder part 21 (i.e., the vicinity of the uppermost portion of the first sheet conveying roller 31) by the second sheet conveying roller 41 is layered on the first sheet member 91 that has passed the uppermost portion of the first sheet conveying roller 31.

The bonding roller 51 rotates couterclockwise in FIG. 1 about the third central axis J3 in synchronization with the rotation of the first sheet conveying roller 31. Like the bonding roller 51, each auxiliary roller 42 also rotates counterclockwise in FIG. 1. Each auxiliary roller 32 rotates clockwise in FIG. 1, like the first sheet conveying roller 31. The first application part 61 is located above the plurality of auxiliary rollers 32 and applies an adhesive onto the first sheet member 91. The second application part 62 is located above the plurality of auxiliary rollers 42 and applies an adhesive onto the second sheet member 92.

The second sheet member 92 that has passed the second sheet conveying roller 41 is layered on the first sheet member 91. The first sheet member 91 and the second sheet member 92 are then sandwiched between the first sheet conveying roller 31 and the bonding roller 51 and bonded to each other. The first sheet conveying roller 31 and the bonding roller 51 constitute a sheet bonding part for bonding the first sheet member 91 and the second sheet member 92 to each other.

Above the cylinder part 21 is a particle filling part 23. The particle filling part 23 includes a particle tank 231 for housing highly absorbent resin particles above the cylinder part 21, and a level sensor 233 provided in the particle tank 231. When the level sensor 233 has detected that the amount of particles in the particle tank 231 is less than or equal to a certain amount, the particle tank 231 is replenished with particles. The particle tank 231 extends approximately parallel to the direction of gravity and has at the lower end a particle filling opening 232 that faces the cylinder outer surface 211. The particle filling opening 232 faces an area of the cylinder part 21 that includes the uppermost portion.

A first cover part 221 and a second cover part 222 are provided around the cylinder part 21. The first cover part 221 extends in the rotation direction of the cylinder part 21 (i.e., counterclockwise in FIG. 1) from the particle filling opening 232 to the lower portion of the cylinder part 21. The first cover part 221 is a half cover part that covers part of the cylinder outer surface 211 on the left side of the cylinder part 21. The second cover part 222 extends in the opposite direction (i.e., clockwise in FIG. 1) to the rotation direction of the cylinder part 21 from the particle filling opening 232 to the lower portion of the cylinder part 21. The second cover part 222 is another half cover part that covers another part of the cylinder outer surface 211 on the right side of the cylinder part 21.

The cylinder outer surface 211 of the cylinder part 21 has a region that is exposed from the first cover part 221 and the second cover part 222 at the lower portion of the cylinder part 21. This region is, as will be described later, a particle supply region 210 in which particles are supplied. The first cover part 221 extends clockwise from the particle supply region 210 along the cylinder outer surface 211, and the second cover part 222 extends counterclockwise from the particle supply region 210 along the cylinder outer surface 211. In the absorbent sheet manufacturing apparatus 1, the configuration including the cylinder part 21, particle filling part 23, the first cover part 221, and the second cover part 222 is a particle supplying apparatus 2. The second cover part 222 may be omitted depending on the design of the particle supplying apparatus 2.

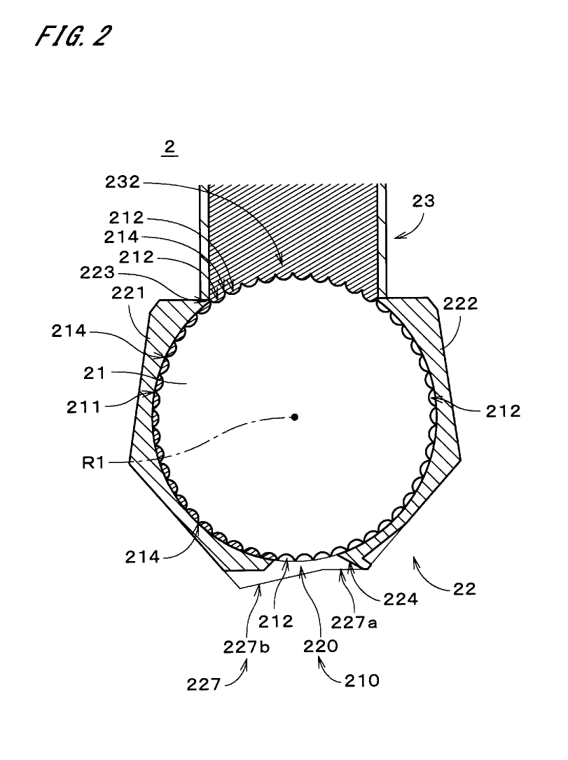

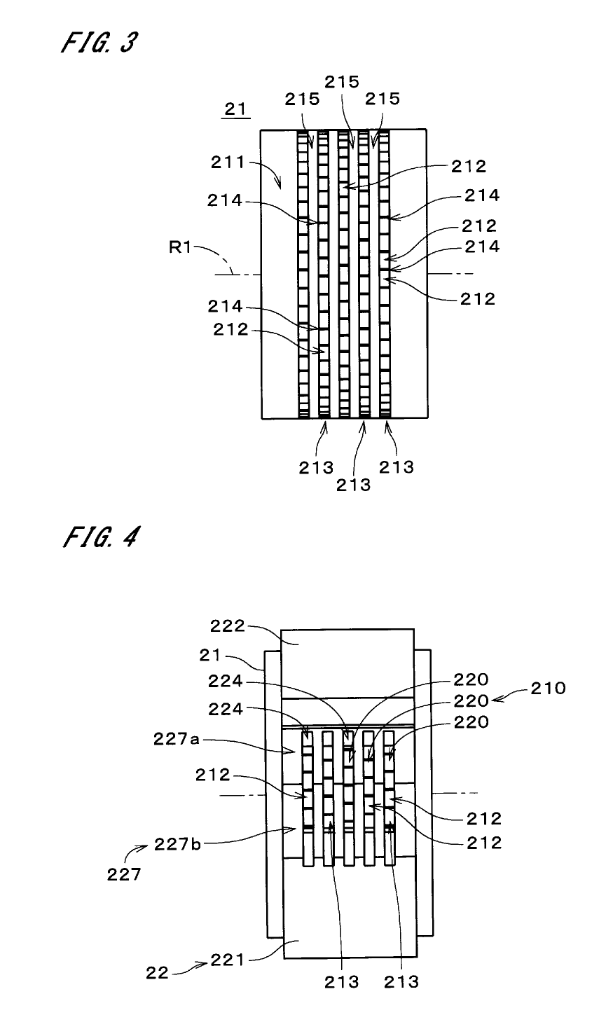

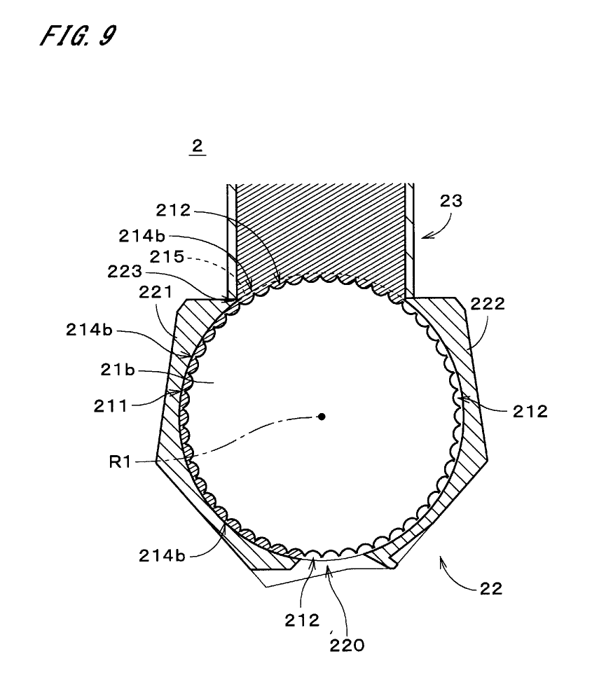

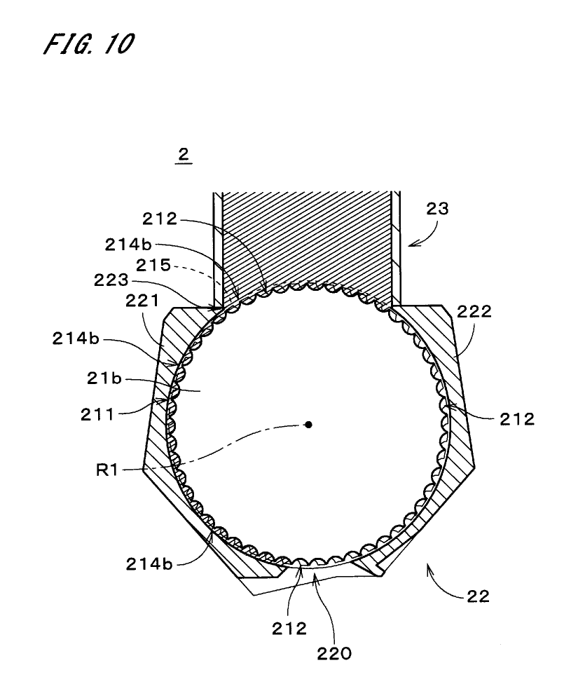

FIG. 2 is an enlarged cross-sectional view of the configuration of the cylinder part 21 and the vicinity of the cylinder part 21. FIG. 2 illustrates a cross-section perpendicular to the cylinder rotation axis R1 and also illustrates an area that is located behind the cross-section (the same applies to FIGS. 9 and 10, which will be described later). FIG. 3 illustrates the cylinder outer surface 211 of the cylinder part 21 as viewed in a direction perpendicular to the cylinder rotation axis R1. In FIG. 2, particles are finely diagonally hatched. Illustration of the first cover part 221 and the second cover part 222 has been omitted from FIG. 3.

As illustrated in FIGS. 2 and 3, the cylinder outer surface 211 of the cylinder part 21 has a plurality of recessed portions 212 which are supply holes at each of a plurality of axial positions, and the recessed portions 212 are arrayed fully circumferentially about the cylinder rotation axis R1 at a fixed recessed-portion pitch. When a plurality of recessed portions 212 that are arranged circumferentially at the same axial position are referred to as a “recessed portion row 213,” the cylinder part 21 includes a plurality of recessed portion rows 213 (i.e., supply hole rows) arranged along the cylinder rotation axis R1 as illustrated in FIG. 3. The cylinder part 21 includes, for example, five recessed portion rows 213. Each recessed portion 212 has, for example, a generally rectangular shape as viewed in a direction perpendicular to the cylinder rotation axis R1. In a cross-section perpendicular to the cylinder rotation axis R1, each recessed portion 212 has a bottom surface having, for example, a generally arc-like shape as illustrated in FIG. 2. The recessed portions 212 may be of various shapes, and for example, each recessed portion 212 may have a generally rectangular shape in a cross-section perpendicular to the cylinder rotation axis R1.

When a portion of the cylinder outer surface 211 that divides two circumferentially adjacent recessed portions 212 is referred to as a “divider 214,” the circumferential positions of dividers 214 differ between two axially adjacent recessed portion rows 213 of the cylinder part 21 in FIG. 3. To be more specific, the dividers 214 extend linearly and parallel to the axial direction. Among the five recessed portion rows 213, three consecutive recessed portion rows 213 consisting of the central recessed portion row 213 and two recessed portion rows 231 on the left of the central recessed portion row 213 have dividers 214 at different circumferential positions. Similarly, three consecutive recessed portion rows 213 consisting of the central recessed portion row 213 and two recessed portion rows on the right of the central recessed portion row 213 have dividers 214 at different circumferential positions. In actuality, two recessed portion rows 213 located on both axial ends have dividers 214 at the same circumferential positions, and two recessed portion rows 213 located on both adjacent sides of the central recessed portion row 213 have dividers 214 at the same circumferential positions. In this way, the plurality of recessed portion rows 213 of the cylinder part 21 in FIG. 3 include three recessed portion rows 213 among which the circumferential positions of the dividers 214 differ. In these three recessed portion rows 213, with respect to one divider 214 in one of the recessed portion rows 213, two respective dividers 214 of the other two recessed portion rows 213 are respectively located one-third and two-thirds of the recessed-portion pitch away from the one divider 214 in the rotation direction. That is, the dividers 214 of the three recessed portion rows 213 as a whole are disposed at equal intervals in the circumferential direction.

As illustrated in FIG. 2, portions of the cylinder part 21 that are covered by the first cover part 221 and the second cover part 222, excluding the recessed portions 212 of the cylinder outer surface 211, are in extremely close proximity to or substantially in contact with the inner surfaces of the first cover part 221 and the second cover part 222. That is, regions 215 (hereinafter, referred to as “inter-row regions 215”) of the cylinder outer surface 211 in FIG. 3 that are located between each pair of adjacent recessed portion rows 213, and the surfaces (outermost surfaces in the radial direction centered on the cylinder rotation axis R1) of the plurality of dividers 214 are substantially in contact with the inner surfaces of the first cover part 221 and the second cover part 222. In the following description, the “cylinder outer surface 211” when simply referred to mainly indicates the “inter-row regions 215.”

FIG. 4 is a bottom view of the lower portions of the cylinder part 21, the first cover part 221, and the second cover part 222, i.e., the particle supply region 210 and the area in the vicinity of the particle supply region 210. As illustrated in FIGS. 2 and 4, the first cover part 221 and the second cover part 222 in the present embodiment constitute an integral member 22 (hereinafter, referred to as a “casing 22”). As illustrated in FIG. 4, the lowermost portion of the casing 22 has a plurality of slits 220 that respectively expose the plurality of recessed portion rows 213. The particle supply region 210 already described is a group of the plurality of slits 220 that correspond respectively to the plurality of recessed portion rows 213. The axial width of each slit 220 is approximately equal to the axial width of a recessed portion 212. In each slit 220, a side surface 224 (hereinafter, referred to as a “particle collision surface 224”) on the right in FIG. 2 (i.e., forward in the rotation direction of the cylinder part 21) is inclined down to the right in FIG. 2 from the vicinity of the cylinder outer surface 211. In other words, the particle collision surface 224 is an inclined surface that is inclined forward in the rotation direction of the cylinder part 21 as the particle collision surface 224 approaches downward in the vertical direction.

A lower end surface 227 of the casing 22 includes a horizontal portion 227a and an inclined portion 227b. The horizontal portion 227a extends approximately horizontally and forward in the rotation direction of the cylinder part 21 from below the vicinity of the lowermost portion of the cylinder part 21. The inclined portion 227b is contiguous with the horizontal portion 227a below the vicinity of the lowermost portion of the cylinder part 21, and extends downward as the inclined portion 227b approaches backward in the rotation direction of the cylinder part 21. In other words, the inclined portion 227b extends downward as the inclined portion 227b approaches toward the first cover part 221 from the vicinity of the particle collision surface 224.

In the absorbent sheet manufacturing apparatus 1, the cylinder part 21 in FIG. 2 rotates at a high speed about the cylinder rotation axis R1, and the plurality of recessed portions 212 in each recessed portion row 213 passing under the particle filling part 23 are successively filled with particles under the force of gravity at the particle filling opening 232. The particles contained in the recessed portions 212 are leveled off along the cylinder outer surface 211 with an edge 223 (edge that extends straight in the axial direction and is hereinafter referred to as a “rear edge 223”) of the first cover part 221 on the rear side in the rotation direction. This allows a certain amount of particles to be retained in the recessed portions 212. At this time, since the plurality of recessed portion rows 213 of the cylinder part 21 include three recessed portion rows 213 among which the circumferential positions of the dividers 214 differ (see FIG. 3), the time at which each divider 214 passes the rear edge 223 that is in contact with the particle filling opening 232 differs among the three recessed portion rows 213.

In the absorbent sheet manufacturing apparatus 1, the outer sides of the recessed portions 212 filled with particles are covered by the first cover part 221 (i.e., the recessed portions 212 are covered from the cylinder outer surface 211 side) until when the recessed portions 212 reach the particle supply region 210 (slits 220) provided in the lower portion of the cylinder part 21. When each recessed portion 212 is passing the particle supply region 210 over the edge of the first cover part 221 in the lower portion of the cylinder part 21, i.e., the front edge of the first cover part 221 in the rotation direction of the cylinder part 21, particles in the recessed portion 212 are emitted to the outside of the cylinder part 21. The particles from the recessed portions 212 are emitted forward in the rotation direction of the cylinder part 21 approximately along the direction of tangent to the cylinder outer surface 211 at the front edge.

Some of the particles emitted at the front edge of the first cover part 221 from the recessed portions 212 are directly supplied onto the first sheet member 91 (see FIG. 1) that is being conveyed under the cylinder part 21 by the first sheet conveying roller 31. Some other particles collide with the particle collision surface 224 and are guided onto the first sheet member 91 by the particle collision surface 224. Some other particles collide with the second sheet member 92 (see FIG. 1), bounce back off the second sheet member 92, and are supplied onto the first sheet member 91. In the particle supplying apparatus 2, the inner side surfaces of the slits 220 of the casing 22 in FIG. 4 that are perpendicular to the axial direction suppress scattering of particles toward positions that are deviated in the axial direction.

In the absorbent sheet manufacturing apparatus 1 in FIG. 1, the first application part 61 applies an adhesive onto a plurality of longitudinally extending strip-like regions (or linear regions) of the first sheet member 91 when the first sheet member 91 is guided to the first sheet conveying roller 31 through the plurality of auxiliary rollers 32. The plurality of strip-like regions have the same axial positions as the axial positions of the plurality of recessed portion rows 213. With this configuration, particles can easily be caught in the plurality of strip regions of the first sheet member 91.

As described above, in the absorbent sheet manufacturing apparatus 1, the cylinder part 21 is rotated in such a way that particles are successively emitted from the plurality of recessed portions 212 and supplied onto the first sheet member 91 either directly or indirectly through the second sheet member 92 or the particle collision surface 224. Each recessed portion 212, after the emission of particles toward the first sheet member 91, passes the particle supply region 210 illustrated in FIG. 1, and with the outer side thereof covered by the second cover part 222, travels to the upper portion of the cylinder part 21 and to the particle filling opening 232 of the particle filling part 23.

On the other hand, the second sheet member 92 is guided to the second sheet conveying roller 41 through the plurality of auxiliary rollers 42. At this time, the second application part 62 applies an adhesive in strips to the same axial positions of the second sheet member 92 as the axial positions of the plurality of strip regions of the first sheet member 91. The second sheet member 92 that has passed the second sheet conveying roller 41 is layered on the first sheet member 91 where particles have been supplied to each strip region.

The first sheet member 91 and the second sheet member 92 are sandwiched between the first roller outer surface 311 of the first sheet conveying roller 31 and the bonding roller outer surface 511 of the bonding roller 51. Both (or only one) of the first sheet conveying roller 31 and the bonding roller 51 are provided with a heater, and annular grooves are formed at positions facing the strip regions of the first sheet member 91. The entire regions of the first sheet member 91 and the second sheet member 92, excluding the plurality of strip regions, are sealed by heat. Consequently, the first sheet member 91 and the second sheet member 92 are bonded to each other. Through the procedure described above, an absorbent sheet is manufactured with strip-like particle existence regions having high-absorbent resin particles dispersed thereon.

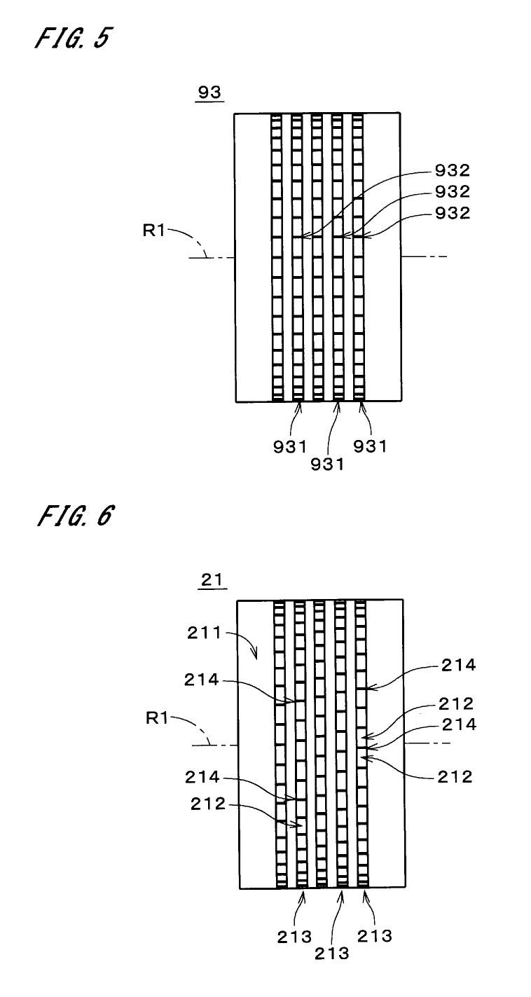

FIG. 5 illustrates a cylinder part 93 according to a comparative example. With the cylinder part 93 of the comparative example, dividers 932 are located at the same circumferential positions among all recessed portion rows 931, and pass the rear edge 223 (see FIG. 2) of the first cover part 221 at the same times among all of the recessed portion rows 931. Thus, an impact that may be caused by particles getting caught between the rear edge 223 and the dividers 932 (impact at the time of particle crashing) will be approximately simultaneously given to all of the recessed portion rows 931, and a strong impact will be given to the cylinder part 93 and the first cover part 221.

In contrast, in the cylinder part 21 in FIG. 3, the plurality of recessed portion rows 213 include three recessed portion rows 213 among which the circumferential positions of the dividers 214 differ. This configuration allows the dividers 214 in the plurality of recessed portion rows 213 to pass the rear edge 223 of the first cover part 221 at dispersed times. It is thus possible to disperse and reduce an impact that may be caused by particles getting caught between the dividers 214 in the plurality of recessed portion rows 213 and the rear edge 223 of the first cover part 221. Consequently, the occurrence of malfunctions due to strong impacts being repeatedly given to the particle supplying apparatus 2 can be reduced.

In addition, a torque necessary to rotate the cylinder part 21 can be reduced. Thus, even when a large number of recessed portion rows 213 are provided, a small-size driver can be used to generate a rotation torque. Moreover, since the three recessed portion rows 213 have dividers 214 at equally spaced circumferential positions, it is possible to appropriately disperse the times at which the dividers 214 pass the rear edge 223 of the first cover part 221 among the plurality of recessed portion rows 213. Thus, the impact to the particle supplying apparatus 2 can more reliably be reduced.

FIG. 6 illustrates another example of the cylinder part 21. Like the cylinder part 21 in FIG. 4, the cylinder part 21 in FIG. 6 includes five recessed portion rows 213. With respect to one divider 214 in one of the recessed portion rows 213, four respective dividers 214 of the other four recessed portion rows 213 are respectively located one-fifth, two-fifth, three-fifth, and four-fifth of the recessed-portion pitch away from the one divider 214 in the rotation direction. That is, all of the recessed portion rows 213 have dividers 214 at different circumferential positions. With this configuration, it is possible to further disperse the times at which the dividers 214 pass the rear edge 223 of the first cover part 221 among the plurality of recessed portion rows 213, and to further disperse and reduce an impact that may be caused by particles getting caught between the dividers 214 in the plurality of recessed portion rows 213 and the rear edge 223 of the first cover part 221. In addition, since all of the recessed portion rows 213 have dividers 214 at equally spaced circumferential positions, the impact to the particle supplying apparatus 2 can more reliably be reduced.

An absorbent sheet with strip-like particle existence regions usually includes three or more strip regions to which particles are applied, and therefore, the cylinder part 21 often includes three or more recessed portion rows 213. From the viewpoint of dispersing the times at which the dividers 214 pass the rear edge 223 of the first cover part 221 among the plurality of recessed portion rows 213, the plurality of recessed portion rows 213 may preferably include at least three recessed portion rows 213 among which the circumferential positions of the dividers 214 differ. That is, the times at which the dividers 214 pass the rear edge 223 may preferably be dispersed into three or more.

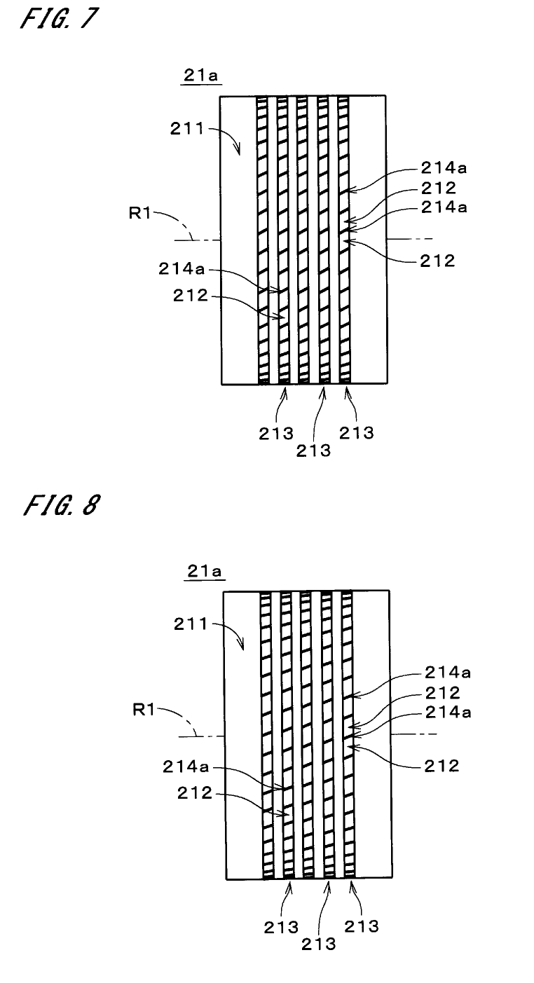

FIG. 7 illustrates a cylinder part 21a according to a second embodiment of the present invention. In the cylinder part 21a in FIG. 7, each divider 214a extends in a direction that is inclined with respect to the cylinder rotation axis R1 on the cylinder outer surface 211. An angle (hereinafter, referred to as an “inclination angle”) that is formed by a divider 214a and the direction parallel to the cylinder rotation axis R1 on the cylinder outer surface 211 may preferably be greater than or equal to 0.5 degrees and less than or equal to 30 degrees. In the present embodiment, all dividers 214a have the same inclination angle. In the cylinder part 21a in FIG. 7, dividers 214a are located at the same circumferential positions among all of the plurality of recessed portion rows 213. The other configuration of the particle supplying apparatus 2, including the cylinder part 21a, is similar to the configuration of the first embodiment.

In the cylinder part 21a in FIG. 7, particles applied to the recessed portions 212 by the particle filling part 23 (see FIG. 2) are leveled off with the rear edge 223 of the first cover part 221 in such a way that a certain amount of particles is retained in the recessed portions 212. At this time, each divider 214a is inclined with respect to the axial direction, whereas the rear edge 223 extends straight in the axial direction. Thus, a plurality of consecutive axial positions of each divider 214a pass the rear edge 223 at different times (it can also be perceived that the divider 214a is in point contact with the rear edge 223). With this configuration, it is possible to disperse and reduce an impact that may be caused by particles getting caught between the dividers 214a in the plurality of recessed portion rows 213 and the rear edge 223 of the first cover part 221. Note that the inclination angle of the dividers 214a may be changed for each recessed portion row 213.

As described previously, from the viewpoint of dispersing the times at which the dividers 214a pass the rear edge 223 among the plurality of recessed portion rows 213, at least three recessed portion rows 213 among the plurality of recessed portion rows 213 may preferably have dividers 214 at different circumferential positions. A cylinder part 21a including such recessed portion rows 213 will now be described.

FIG. 8 illustrates another example of the cylinder part 21a. In the cylinder part 21a in FIG. 8, each divider 214a extends linearly in a direction that is inclined with respect to the cylinder rotation axis R1 on the cylinder outer surface 211, as in the cylinder part 21a in FIG. 7. With respect to one divider 214a in one of the recessed portion rows 213, four respective dividers 214a of the other four recessed portion rows 213 are respectively located one-fifth, two-fifth, three-fifth, and four-fifth of the recessed-portion pitch (the distance in the circumferential direction between the centers of the recessed portions 212) away from the one divider 214a in the rotation direction. That is, all of the recessed portion rows 213 have dividers 214a at different circumferential positions. With this configuration, the times at which the dividers 214a, which extend in the direction inclined with respect to the axial direction, pass the rear edge 223 can be dispersed among the plurality of recessed portion rows 213. Consequently, it is possible to further disperse and reduce an impact that may be caused by particles getting caught between the dividers 214a in the plurality of recessed portion rows 213 and the rear edge 223 of the first cover part 221. In addition, since all of the recessed portion rows 213 have dividers 214a at equally spaced circumferential positions, the impact to the particle supplying apparatus 2 can more reliably be reduced.

FIG. 9 is an enlarged cross-sectional view of a configuration of a cylinder part 21b according to a third embodiment of the present invention and the vicinity of the cylinder part 21b. In the particle supplying apparatus 2 in FIG. 9, the vertices of dividers 214b having an angular cross-sectional shape, i.e., the outermost surfaces of the divider 214b in the radial direction, are located closer to the cylinder rotation axis R1 than the inter-row regions 215 located between the plurality of recessed portion rows 213 are on the cylinder outer surface 211. In other words, the surfaces of the divider 214 are located inwardly of the cylinder outer surface 211 toward the cylinder rotation axis R1. The other configuration of the particle supplying apparatus 2 in FIG. 9 is similar to the configuration of the particle supplying apparatus 2 in FIG. 2, and therefore, constituent elements that are identical to those in FIG. 2 are given by the same reference numerals.

In the cylinder part 21b in FIG. 9, the dividers 214b extend straight in the axial direction, and particles applied to the recessed portions 212 by the particle filling part 23 are leveled off with the rear edge 223 of the first cover part 221 that extends straight in the axial direction. At this time, a slight gap between the surfaces of the dividers 214b and the rear edge 223 (the inner surface of the first cover part 221) prevents particles that are caught between the dividers 214b and the rear edge 223 from being crushed completely. It is thus possible to reduce an impact that may be caused by particles getting caught between the dividers 214b in the plurality of recessed portion rows 213 and the rear edge 223 of the first cover part 221. The width of the gap between the surfaces of the dividers 214b and the inner surface of the first cover part 221 may preferably be greater than or equal to 0.01 millimeters (mm) and less than or equal to 0.5 mm and may, for example, be 0.2 mm. The width of the gap may preferably be smaller than the average diameter of particles.

FIG. 10 illustrates another example of the cylinder part 21b. The cylinder part 21b in FIG. 10 differs from the cylinder part 21b in FIG. 9 only in that the dividers 214b are inclined with respect to the axial direction. With the dividers 214b inclined with respect to the axial direction, a plurality of consecutive positions of each divider 214b along the axial direction pass the rear edge 223 at different times. With this configuration, it is possible to disperse an impact that may be caused by particles getting caught between the dividers 214b in the plurality of recessed portion rows 213 and the rear edge 223 of the first cover part 221. This, in combination with the effect of reducing the impact in the presence of a small gap between the surfaces of the dividers 214b and the inner surface of the first cover part 221, results in a considerable reduction in the impact to the particle supplying apparatus 2.

In the cylinder parts 21b in FIGS. 9 and 10, the plurality of recessed portion rows 213 may have dividers 214b at the same circumferential positions. A preferable cylinder part 21b may include three or more recessed portion rows 213, and the three or more recessed portion rows 213 may include at least three recessed portion rows 213 among which the circumferential positions of corresponding dividers 214 differ. With this configuration, the impact to the particle supplying apparatus 2 can considerably be reduced. In a more preferable cylinder part 21b, all of the plurality of recessed portion rows 213 may have dividers 214b at different circumferential positions. This configuration further reduces the impact to the particle supplying apparatus 2.

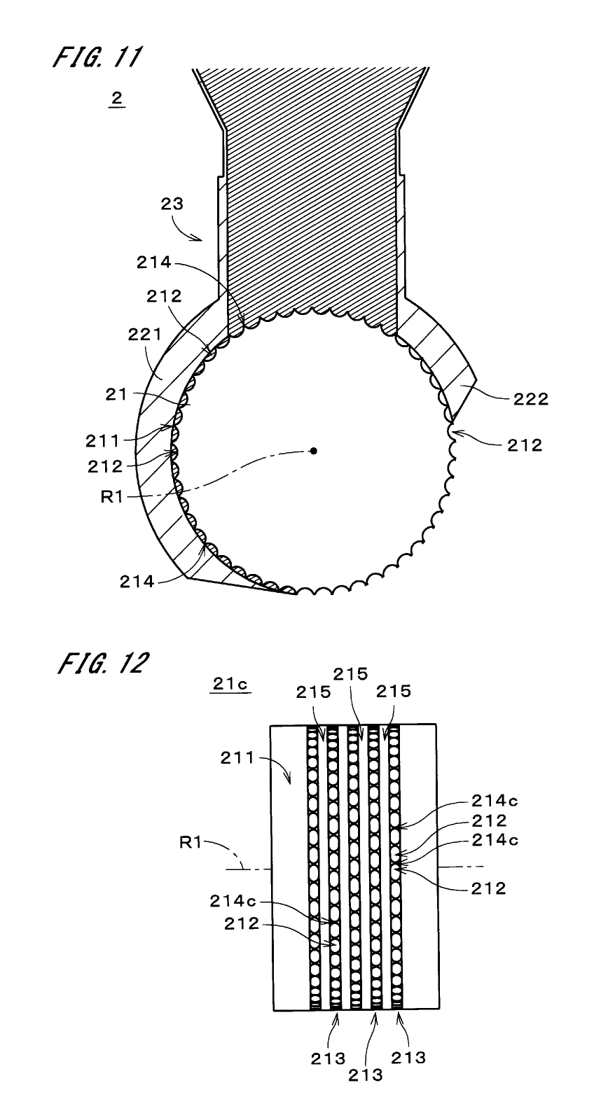

The above-described particle supplying apparatus 2 may be modified in various ways. For example, the first cover part 221 and the second cover part 222 may be two separate independent members as illustrated in FIG. 11. In the absorbent sheet manufacturing apparatus 1 including the particle supplying apparatus 2 in FIG. 11, the cylinder part 21 and the first sheet conveying roller 31 may be disposed in closer proximity to each other.

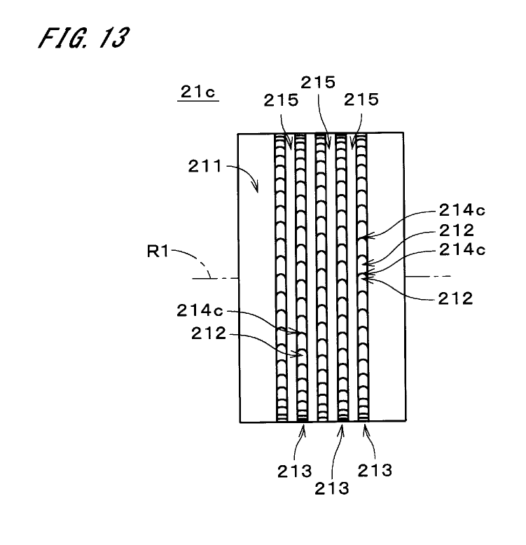

As illustrated in FIG. 12 or 13, a cylinder part 21c may use dividers 214c, each having an arc-shaped edge along the axial direction. With such a divider 214c, a time at which a position of the divider 214c in the vicinity of the center in the axial direction passes the rear edge 223 is different from the time at which a position of the divider 214c in the vicinity of each end in the axial direction passes the rear edge 223. This configuration considerably reduces the impact to the particle supplying apparatus 2, in combination with the configuration in which the cylinder part 21c includes at least three recessed portion rows 213 among which the circumferential positions of corresponding dividers 214c differ, or in combination with the configuration in which the surfaces of the dividers 214c are located closer to the cylinder rotation axis R1 than the inter-row regions 215 are.

In the absorbent sheet manufacturing apparatus 1, if the recessed portions 212 can be filled with particles in sufficiently high density, the particle filling opening 232 does not necessarily have to face the area of the cylinder part including the uppermost portion. For example, the particle filling opening 232 may be located to face a position of the cylinder outer surface 211 which is slightly shifted forward or backward of the uppermost portion in the rotation direction.

In the particle supplying apparatus 2, particles of a deodorant material may be supplied onto the first sheet member 91, instead of particles of an absorbent material. An absorbent-article sheet member manufacturing apparatus including such a particle supplying apparatus 2 manufactures deodorant sheets. Examples of particles of the deodorant material include particles of activated carbon, silica, alumina, zeolite, ion-exchange resins, and molecular sieve. The deodorant sheets are usable in absorbent articles such as disposable diapers and absorbent pads for light incontinence.

The configurations of the above-described preferred embodiments and variations may be appropriately combined as long as there are no mutual inconsistencies.

While the invention has been shown and described in detail, the foregoing description is in all aspects illustrative and not restrictive. It is therefore to be understood that numerous modifications and variations can be devised without departing from the scope of the invention.

### REFERENCE SIGNS LIST

2 Particle supplying apparatus
21, 21a to 21c Cylinder part
23 Particle filling part
91 First sheet member
211 Cylinder outer surface
212 Recessed portion
213 Recessed portion row
214, 214a to 214c Divider
215 Inter-row region
221 First cover part
232 Particle filling opening
R1 Cylinder rotation axis

1. A particle supplying apparatus for supplying particles of an absorbent material or a deodorant material onto a sheet member, comprising:
a cylinder part of a generally cylindrical shape that rotates about a rotation axis pointing in a horizontal direction and has a plurality of recessed portion rows arranged along said rotation axis, each of said plurality of recessed portion rows being a plurality of recessed portions arrayed at a fixed pitch in a circumferential direction in an outer surface of said cylinder part;
a particle filling part for housing particles of an absorbent material or a deodorant material above said cylinder part and successively filling said plurality of recessed portions in each of said plurality of recessed portion rows with particles at a particle filling opening that opposes said outer surface of said cylinder part; and
a half cover part that extends in a rotation direction from said particle filling opening to a lower portion of said cylinder part and covers part of said outer surface of said cylinder part to allow particles to be successively emitted in the vicinity of a front edge of said half cover part in said rotation direction from said plurality of recessed portions toward a sheet member that is being conveyed under said cylinder part,
wherein said plurality of recessed portion rows are three or more recessed portion rows, and
when a portion of said cylinder part that divides two recessed portions adjacent to each other in said circumferential direction is referred to as a divider, said plurality of recessed portion rows include at least three recessed portion rows among which positions of dividers in said circumferential direction differ.

2. The particle supplying apparatus according to claim 1, wherein
the positions of dividers in said circumferential direction differ among all of said plurality of recessed portion rows.

3. The particle supplying apparatus according to claim 1, wherein
each divider extends in a direction inclined with respect to said rotation axis on said outer surface of said cylinder part.

4. The particle supplying apparatus according to claim 1, wherein
each divider has a surface that is located closer to said rotation axis than regions between said plurality of recessed portion rows are on said outer surface of said cylinder part.

5. A particle supplying apparatus for supplying particles of an absorbent material or a deodorant material onto a sheet member, comprising:
a cylinder part of a generally cylindrical shape that rotates about a rotation axis pointing in a horizontal direction and has a plurality of recessed portion rows arranged along said rotation axis, each of said plurality of recessed portion rows being a plurality of recessed portions arrayed at a fixed pitch in a circumferential direction in an outer surface of said cylinder part;
a particle filling part for housing particles of an absorbent material or a deodorant material above said cylinder part and successively filling said plurality of recessed portions in each of said plurality of recessed portion rows with particles at a particle filing opening that opposes said outer surface of said cylinder part; and
a half cover part that extends in a rotation direction from said particle filling opening to a lower portion of said cylinder part and covers part of said outer surface of said cylinder part to allow particles to be successively emitted in the vicinity of a front edge of said half cover part in said rotation direction from said plurality of recessed portions toward a sheet member that is being conveyed under said cylinder part,
wherein, when a portion of said cylinder part that divides two recessed portions adjacent to each other in said circumferential direction is referred to as a divider, each divider extends in a direction inclined with respect to said rotation axis on said outer surface of said cylinder part.

6. The particle supplying apparatus according to claim 5, wherein
said each divider has a surface that is located closer to said rotation axis than a region between said plurality of recessed portion rows is on said outer surface of said cylinder part.

7. A particle supplying apparatus for supplying particles of an absorbent material or a deodorant material onto a sheet member, comprising:
a cylinder part of a generally cylindrical shape that rotates about a rotation axis pointing in a horizontal direction and has a plurality of recessed portion rows arranged along said rotation axis, each of said plurality of recessed portion rows being a plurality of recessed portions arrayed at a fixed pitch in a circumferential direction in an outer surface of said cylinder part;
a particle filling part for housing particles of an absorbent material or a deodorant material above said cylinder part and successively filling said plurality of recessed portions in each of said plurality of recessed portion rows with particles at a particle filling opening that opposes said outer surface of said cylinder part; and
a half cover part that extends in a rotation direction from said particle filling opening to a lower portion of said cylinder part and covers part of said outer surface of said cylinder part to allow particles to be successively emitted in the vicinity of a front edge of said half cover part in said rotation direction from said plurality of recessed portions toward a sheet member that is being conveyed under said cylinder part,
wherein, when a portion of said cylinder part that divides two recessed portions adjacent to each other in said circumferential direction is referred to as a divider, each divider has a surface that is located closer to said rotation axis than a region between said plurality of recessed portion rows is on said outer surface of said cylinder part.

8. The particle supplying apparatus according to claim 2, wherein
each divider extends in a direction inclined with respect to said rotation axis on said outer surface of said cylinder part.

9. The particle supplying apparatus according to claim 2, wherein
each divider has a surface that is located closer to said rotation axis than regions between said plurality of recessed portion rows are on said outer surface of said cylinder part.

10. The particle supplying apparatus according to claim 3, wherein
each divider has a surface that is located closer to said rotation axis than regions between said plurality of recessed portion rows are on said outer surface of said cylinder part.

11. The particle supplying apparatus according to claim 8, wherein
each divider has a surface that is located closer to said rotation axis than regions between said plurality of recessed portion rows are on said outer surface of said cylinder part.
